(19) United States
(12) Patent Application Publication
(10) Pub. No.: US 2014/0018612 A1
(43) Pub. Date: Jan. 16, 2014

(54) TRACK-TYPE SEX AID

(71) Applicant: HAN CHING LIN, TAINAN CITY (TW)

(72) Inventor: HAN CHING LIN, TAINAN CITY (TW)

(21) Appl. No.: 13/545,244

(22) Filed: Jul. 10, 2012

(51) Int. Cl. A61F 5/00

(52) U.S. Cl. 600/38

(57) ABSTRACT

A track-type sex aid includes a track having an outer periphery and an inner periphery opposite to the outer periphery. The track includes a plurality of protrusions protruding beyond the outer periphery of the track. An annular transmission device includes first and second wheels rotatably mounted in a space defined by the inner periphery of the track. The first wheel includes a shaft directly or indirectly coupled to a driving shaft of a power output mechanism of the annular transmission device, with the first wheel directly or indirectly driving the track.

### BACKGROUND OF THE INVENTION

The present invention relates to a sex aid and, more particularly, to a track-type sex aid.

Food and sex are the most important desires of the human. Adults having a sexual relationship are often healthier than singles. Sexual desire is often an embarrassing topic to singles. However, sexual crimes and similar problems could arise if the sexual desires of singles are not properly treated. Although sex workers are an option, the costs include high expense, risks of infection of sexually transmitted diseases, such as syphilis, gonorrhoea, and aids, and violation of conventions. Masturbation, which was deemed as an unhealthy behavior, is another option that is healthier and safer according to recent medical researches. There are more sex aids available in the market as the sexual concept becomes open. Not even singles, couples can have more pleasure in sex by using sex aids.

There are sex aids for males as well as for females. Vibrators are a common sex aid for females. Currently available vibrators generally include a vibration motor to provide reciprocating vibrations. However, the mechanical reciprocating vibrations can not provide the body of a female user with soft, comfortable touch. Although the frequency of the vibration motor can be adjusted, there is no comparison to the touch of fingers or a male sex organ. Further, the surface of the vibrator is too smooth to create an exciting sensation at the vagina of the user, particularly the sensitive clitoris.

### BRIEF SUMMARY OF THE INVENTION

To solve the disadvantages of the prior art, the present invention provides a track-type sex aid including a track having an outer periphery and an inner periphery opposite to the outer periphery. The track includes a plurality of protrusions protruding beyond the outer periphery of the track. An annular transmission device includes first and second wheels rotatably mounted in a space defined by the inner periphery of the track. The first wheel includes a shaft directly or indirectly coupled to a driving shaft of a power output mechanism of the annular transmission device, with the first wheel directly or indirectly driving the track.

Since the track moves along an annular path instead of a rectilinear path, a portion of the annular movement is linear motion similar to movement of fingers or a male sex organ in ergonomics. Thus, a female using the sex aid can obtain a sensation at the skin and the sense organs similar to real feeling resulting from the touch by fingers or a male sex organ. Furthermore, the protrusions on the track create exciting sensations by the continuously arranged protrusions and recesses between the protrusions while the track moves along the annular path. Thus, a frictional exciting effect can be more easily obtained at the vagina of the user, particularly at the more sensitive clitoris.

The present invention will become clearer in light of the following detailed description of illustrative embodiments of this invention described in connection with the drawings.

### DESCRIPTION OF THE DRAWINGS

FIG. 1 shows a perspective view of a track-type sex aid according to the present invention.

FIG. 2 shows a perspective view of an annular transmission device and a track of the track-type sex aid according to the present invention.

FIG. 3 shows an exploded, perspective view of the annular transmission device and the track of the track-type sex aid according to the present invention.

FIG. 4 shows a side view of another example of the annular transmission device and the track of the track-type sex aid according to the present invention.

FIG. 5 shows a perspective view illustrating remote control of the track-type sex aid according to the present invention.

### DETAILED DESCRIPTION OF THE INVENTION

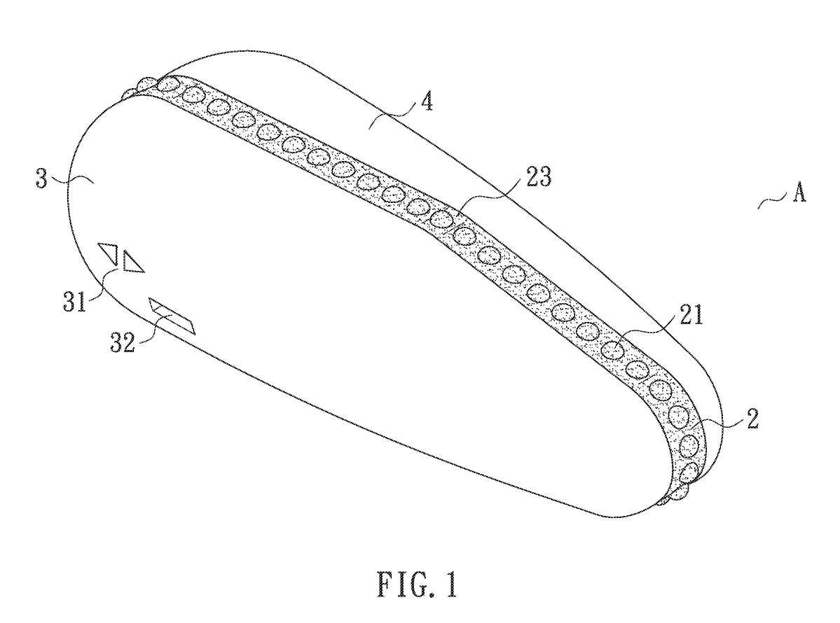

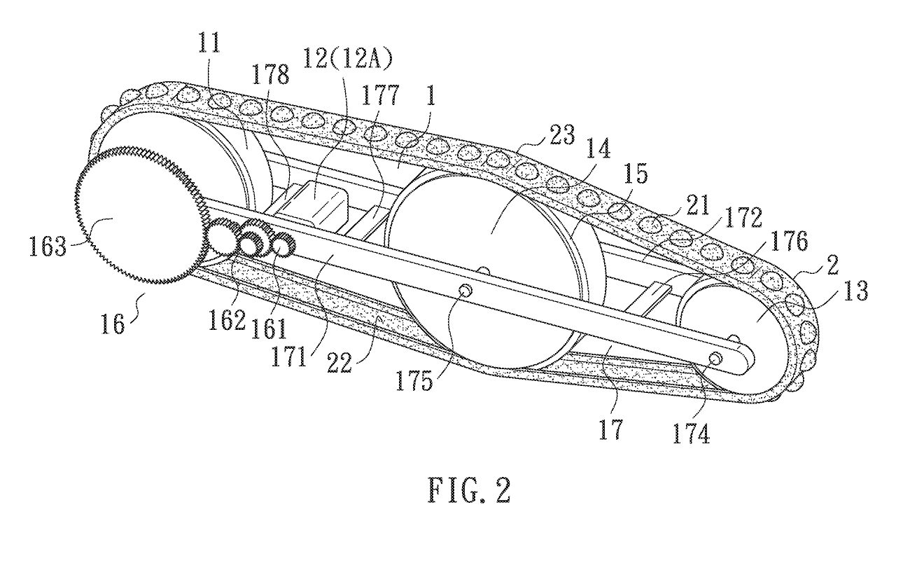

With reference to FIGS. 1 and 2, a track-type sex aid A according to the present invention includes a track 2 and an annular transmission device 1. The track 2 includes an outer periphery and an inner periphery opposite to the outer periphery. The track 2 includes a plurality of protrusions 21 protruding beyond the outer periphery of the track 2. The annular transmission device 1 includes first and second wheels 11 and 13 rotatably mounted in a spaced defined by the inner periphery of the track 2. The annular transmission device 1 further includes a power output mechanism 12 having a driving shaft 164. The first wheel 11 includes a shaft 111 directly or indirectly coupled to the driving shaft 164 of the power output mechanism 12. Thus, the first wheel 11 directly or indirectly drives the track 2.

Since the track 2 moves along an annular path instead of a rectilinear path, a portion of the annular movement is linear motion similar to movement of fingers or a male sex organ in ergonomics. Thus, a female using the sex aid A can obtain a sensation at the skin and the sense organs similar to real feeling resulting from the touch by fingers or a male sex organ. Furthermore, the protrusions 21 on the track 2 create exciting sensations by the continuously arranged protrusions 21 and recesses between the protrusions 21 while the track 2 moves along the annular path. Thus, a frictional exciting effect can be more easily obtained at the vagina of the user, particularly at the more sensitive clitoris.

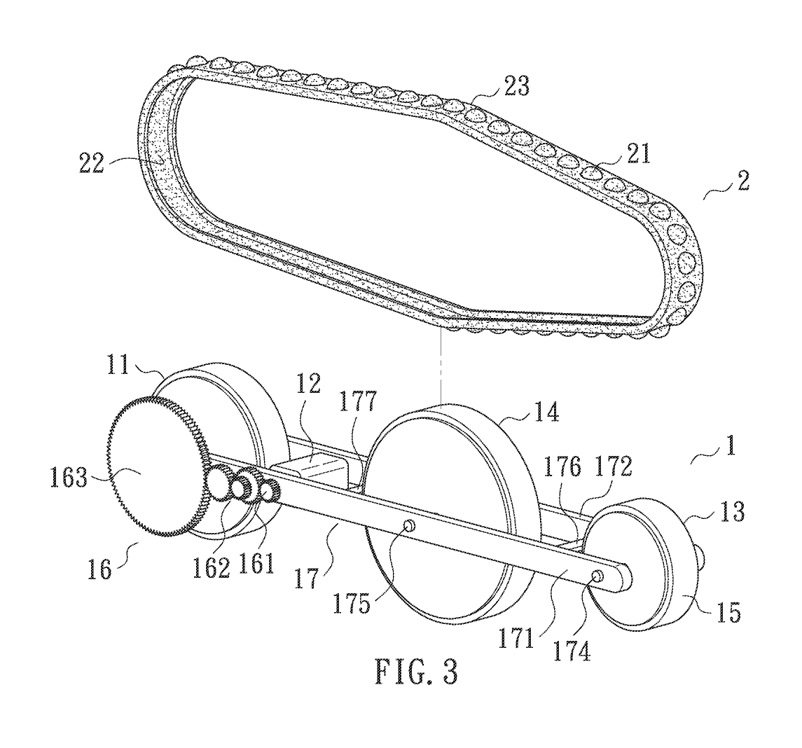

With reference to FIGS. 2 and 3, in the preferred form shown, the first and second wheels 11 and 13 are mounted to the inner periphery of the track 2, with the track 2 being substantially an ellipsoid similar to a male sex organ, allowing the sex aid A to easily enter the vagina of the user. To provide the user with more pleasure, the annular transmission device 2 further includes a third wheel 14 mounted between the first and second wheels 11 and 13. The third wheel 14 is mounted in the space defined by the inner periphery of the track 2 and is in direct or indirect contact with the inner periphery of the track 2. The third wheel 14 has an outer diameter larger than an outer diameter of the first wheel 11 and larger than an outer diameter of the second wheel 13. Thus, the track 2 has a bulged portion 23 for massaging and exciting the sensitive portions of the vagina of the user.

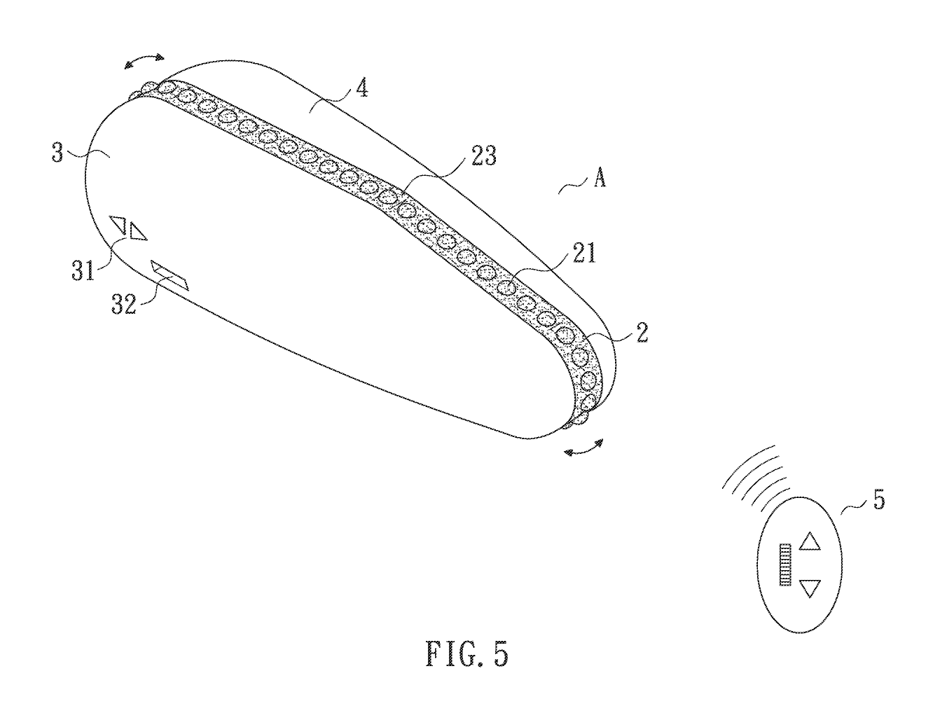

With reference to FIGS. 1 and 5, to provide a complete appearance and to allow easy grip, the sex aid A further includes first and second housing halves 3 and 4. The first and second housing halves 3 and 4 are coupled to each other to form a housing, with the housing receiving the annular transmission device 1. An annular gap is formed between the first and second housing halves 3 and 4, and the track 2 is mounted in the annular gap. The protrusions 21 on the track 2 protrude beyond an outer surface of the housing for contacting with the vagina of the user.

With reference to FIGS. 1 and 5, the first housing half 3 includes a control portion 31 capable of controlling the speed and moving direction of the track 2. The sex aid A can further include a remote control 5, and the control portion 31 includes a signal receiver, with the signal receiver receiving control signals from the remote control 5 to control the speed and moving direction of the track 2. Thus, the user can have more pleasure by different speeds and by changing the moving direction of the track 2. Not only suitable for females, a couple can have more pleasure in sex by using the sex aid A. The first half 3 can include a charging receptacle 32. In the form shown, the charging receptacle 32 is an USB (universal serial bus) charging receptacle 32.

With reference to FIGS. 2 and 3, the annular transmission device 1 includes a frame 17 mounted in the spaced defined by the inner periphery of the track 2 for rotatably mounting the first, second and third wheels 11, 13, and 14. The frame 17 includes first and second beams 171 and 172 on opposite sides of the first and second wheels 11 and 13. A first axle 173 is rotatably mounted between the first and second beams 171 and 172 and extends through the first wheel 11. A second axle 174 is rotatably mounted between the first and second beams 171 and 173 and extends through the second wheel 13. To make the frame 17 firmer, first, second, and third supporting rods 176, 177, and 178 are fixed between the first and second beams 171 and 172. Instead of using the frame 17 to rotatably mount the first, second, and third wheels 11, 13, and 14 in the space defined by the inner periphery of the track 2, a casing can be mounted around the first, second, and third wheels 11, 13, and 14, while the first, second, and third wheels 11, 13, and 14 are rotatably mounted in the spaced defined by the inner periphery of the track 2.

With reference to FIGS. 2 and 3, in addition to the frame 17 for reliably positioning the first, second, and third wheels 11, 13, and 14, the inner periphery of the track 2 includes an annular groove 22. Each of the first, second, and third wheels 11, 13, and 14 includes an annular flange 15, with the annular flanges 15 of the first, second, and third wheels 11, 13, and 14 received in the annular groove 22. The track 2 can be detached and washed, and the track 2 is made of soft silicone rubber for easy washing. Each component of the annular transmission device 1 is processed to be waterproof. Thus, the sex aid A can be cleaned with water to maintain hygiene, avoiding bacterial infection.

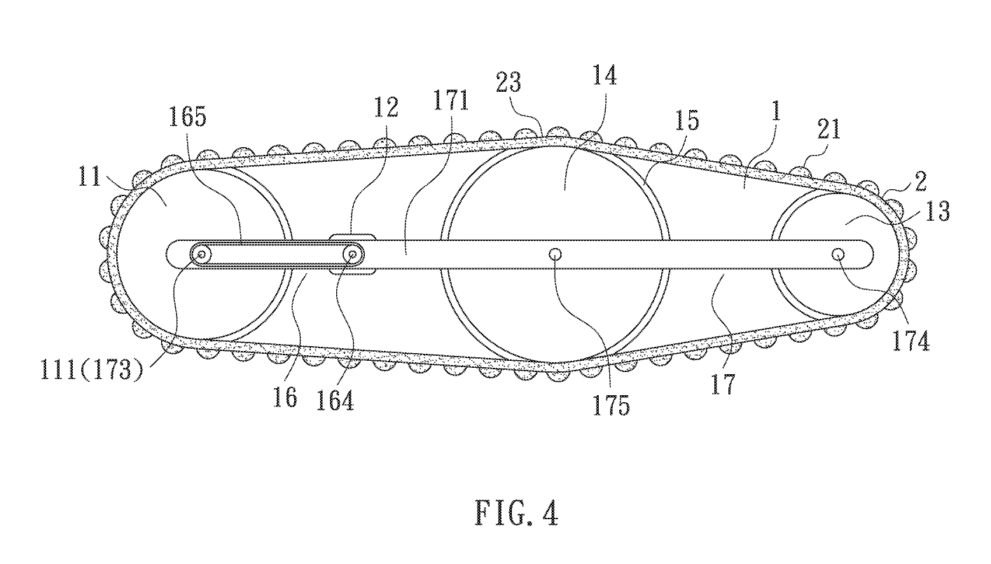

With reference to FIGS. 2 and 3, the shaft 111 of the first wheel 11 is indirectly coupled to the driving shaft 164 of the power output mechanism 12. Specifically, a transmission unit 16 is mounted between the shaft 111 of the first wheel 11 and the driving shaft 164 of the power output mechanism 12. In the example shown in FIGS. 2 and 3, the power output mechanism 12 is a motor 12A. The transmission unit 16 includes a motor gear 161 coupled to the driving shaft 164 of the motor 12A and an axle gear 163 coupled to the first axle 173, with the motor gear 161 directly or indirectly coupled to the axle gear 163. In the example shown in FIGS. 2 and 3, the motor gear 161 is indirectly coupled to the axle gear 163 via a follower gear 162, allowing joint rotation of the motor gear 161 and the axle gear 163. In another example shown in FIG. 4, the transmission unit 16 includes an endless belt 165 directly or indirectly mounted around the driving shaft 164 and the first axle 173, allowing joint rotation of the driving shaft 164 and the first axle 173. Since the motor 12A drives the first wheel 11 by using the endless belt 165, noise resulting from friction between gears can be avoided.

Although specific embodiments have been illustrated and described, numerous modifications and variations are still possible without departing from the essence of the invention. The scope of the invention is limited by the accompanying claims.

1. A track-type sex aid comprising:
a track including an outer periphery and an inner periphery opposite to the outer periphery, with the inner periphery of the track defining a space, with the track including a plurality of protrusions protruding beyond the outer periphery of the track, and
an annular transmission device including first and second wheels rotatably mounted in the space defined by the inner periphery of the track, with the annular transmission device further including a power output mechanism having a driving shaft, with the first wheel including a shaft directly or indirectly coupled to the driving shaft of the power output mechanism, with the first wheel directly or indirectly driving the track.

2. The track-type sex aid as claimed in claim 1, with the annular transmission device further including a third wheel mounted between the first and second wheels, with the third wheel mounted in the space defined by the inner periphery of the track and being in direct or indirect contact with the inner periphery of the track, with the third wheel having an outer diameter larger than an outer diameter of the first wheel and larger than an outer diameter of the second wheel.

3. The track-type sex aid as claimed in claim 1, further comprising: first and second housing halves, with the first and second housing halves coupled to each other to form a housing, with the housing receiving the annular transmission device, with an annular gap formed between the first and second housing halves, with the track mounted in the annular gap, with the plurality of protrusions protruding beyond an outer surface of the housing.

4. The track-type sex aid as claimed in claim 3, with one of the first and second housing halves including a control portion.

5. The track-type sex aid as claimed in claim 4, further comprising: a remote control, with the control portion including a signal receiver, with the signal receiver receiving control signals from the remote control.

6. The track-type sex aid as claimed in claim 1, with a frame mounted in the space defined by the inner periphery of the track, with the frame including first and second beams on opposite sides of the first and second wheels, with a first axle rotatably mounted between the first and second beams and extending through the first wheel, with a second axle rotatably mounted between the first and second beams and extending through the second wheel.

7. The track-type sex aid as claimed in claim 1, with the inner periphery of the track including an annular groove.

8. The track-type sex aid as claimed in claim 7, with each of the first and second wheels including an annular flange, with the annular flanges of the first and second wheels received in the annular groove.

9. The track-type sex aid as claimed in claim 1, with the shaft of the first wheel indirectly coupled to the driving shaft of the power output mechanism, with a transmission unit mounted between the shaft of the first wheel and the driving shaft of the power output mechanism.
